(19) United States
(12) Patent Application Publication
(10) Pub. No.: US 2026/0002326 A1
(43) Pub. Date: Jan. 1, 2026

(54) PROGRESSIVELY FORMED FIBROUS STRUCTURES, AND METHOD AND TOOLING FOR FORMATION THEREOF

(71) Applicant: THE PROCTER & GAMBLE COMPANY, Cincinnati, OH (US)

(72) Inventors: David Andrew DALTON, Mason, OH (US); Benjamin Jacob CLARE, Cincinnati, OH (US); Andrew Joseph HORTON, Middletown, OH (US); Xiao JIANG, West Chester, OH (US); Robert Earl MAGNESS, Lebanon, OH (US); Mark William SHIRES, Brookville, IN (US); Paul Thomas WEISMAN, Cincinnati, OH (US)

(21) Appl. No.: 19/252,880

(22) Filed: Jun. 27, 2025

Related U.S. Application Data
(60) Provisional application No. 63/665,295, filed on Jun. 28, 2024.

(51) Int. Cl. D21J 7/00; A61F 13/20; B29C 33/38; B29C 41/10; B29K 1/00; B33Y 80/00

(52) U.S. Cl. CPC D21J 7/00; A61F 13/2097; B29C 41/10; B29C 33/3814; B29K 2001/00; B33Y 80/00

(57) ABSTRACT

A forming tool for agglomerating thereon fibers from a slurry, the tool having a first end, a second end, a longitudinal axis, a forming surface and a non-forming surface, wherein the forming surface is perforated with a pattern of fluid passage ports extending from the forming surface to the non-forming surface; wherein the pattern of fluid passage ports demarks a surface area of the forming surface.

### CROSS REFERENCE TO RELATED APPLICATIONS

This application claims the benefit, under 35 U.S.C. § 119 (e), to U.S. Provisional Application No. 63/665,295 filed Jun. 28, 2024, the entire disclosure of which is fully incorporated by reference herein.

### BACKGROUND

Forming three-dimensional (3D) structures from fiber material (such as cellulose pulp fibers) is a developing field. Concerns for sustainability have driven many efforts to use cellulose pulp fiber as a material from which to form 3D structures for various applications, in which such structures might otherwise be molded of plastics. This is due to the fact that many types of plastics are deemed by regulatory bodies and/or consumers to cause sustainability concerns, because, for example, they may not be easily recycled, or biodegradable within desirably short times; they may be deemed to contribute to environmental pollution; and they are manufactured from petroleum, which may be deemed not to be a sustainable resource.

One known technique for forming structures of cellulose fiber involves creating a slurry of cellulose pulp fibers suspended in a carrier fluid (e.g., water), inundating a first forming tool having a shaped, porous first forming surface with the slurry, and drawing or pumping the slurry through the first forming surface. The shaped first forming surface will generally reflect the desired shape and configuration of a resulting formed agglomeration of fibers. Pores or ports opening at the first forming surface are of a size that is suitably small enough to prevent passage of any substantial quantity of the fibers through the first tool, such that carrier fluid is drawn or pumped through the first tool and a majority of the fibers in the slurry are not. In effect, the first tool filters the fibers from the slurry, collects an agglomeration of the fibers on the first forming surface, and allows carrier fluid to pass therethrough. Remaining carrier fluid may be further removed from the formed agglomeration, via pressure exerted by a second forming tool, urged along a forming direction against the first forming tool. The second forming tool may have a configuration and second forming surface that mates or cooperates with the first forming surface, with a suitable clearance therebetween, to express carrier fluid from the agglomeration, further shape the agglomeration and compress and densify the fibers, impart a second formed surface, and thereby create a resulting structure formed of the fibers, as the first and second forming tools are urged together along the forming direction. If desired, heating energy may be provided to one or both of the first forming tool and second forming tool to cause evaporation of carrier fluid and/or impart a set to the fibers, and thereby add stiffness and strength to the structure.

The technique described above is currently used to form structures such as cups, trays, packaging materials, and a variety of other products.

A limitation of this technique is that it cannot be used efficiently to form a structure with 3D features that include facing parallel walls or surfaces, such as, for example, 3D rectangular or cylindrical protrusions, channels, tubes, pockets, recesses, etc. This is a matter of geometry. For such features, there will be no formation direction along which single first and second forming tools with respective parallel forming surfaces reflecting such features may be brought together, to effect compression of each/all of the facing parallel walls or surfaces, via single-step, single-direction motion. Further, urging respective forming tools together where the tools have facing forming surfaces parallel with the direction the tools are brought together (forming direction) will cause defects in the desired pulp formation, resulting from shearing and friction between the respective forming surfaces and the pulp agglomeration. Theoretically, a plurality of second tools and associated mechanisms may be configured to effect compression of the pulp agglomeration against the first tool along varying directions, but this would introduce substantial (and potentially, cost prohibitive) complexity and inefficiency to the equipment and the process.

Compromise may be made. Where acceptable, desired 3D features, and associated first and second forming tools, can be designed so as not to have facing walls or surfaces that are parallel, but rather, all respectively facing walls or surfaces are angled with respect to each other and with respect to a forming direction. If the angle with respect to the forming direction is too small, however, bringing the first and second tools together along the forming direction will substantially displace portions of the still-paste-like pulp agglomeration, via shearing friction, imparting defects to the formed structure. For this reason, in industry practice, forming tools typically do not include forming surfaces having any substantial dimension measured parallel the forming direction, that form angles with the forming direction that are less than about 7 degrees.

Due to concerns for sustainability noted above, however, it is becoming desirable to form more types of structures of cellulose fiber for an increasing variety of applications. Accordingly, opportunity is present for advancement of techniques for forming structures of cellulose fiber, and in one particular, for forming structures with features that have facing, near-parallel or parallel walls or surfaces. In one particular but non-limiting example, opportunity for development of nearly-cylindrical or cylindrical tampon applicator components molded of cellulose fiber, as an alternative to plastic, is present.

### BRIEF DESCRIPTION OF THE FIGURES

FIG. 1 is a schematic longitudinal, vertical section view of an example of a first forming tool with associated equipment, depicted during operation of a process.

FIG. 2 is a schematic longitudinal, vertical section view of an example of a first forming tool.

FIG. 3 is a schematic perspective view of a portion of an example of a first forming tool, illustrating a portion of a pattern of fluid passage ports.

FIG. 4A is a schematic lateral section view of a portion of a wall of an example of a first forming tool, illustrating features of fluid passage ports.

FIG. 4B is a schematic magnified lateral section view of a portion of a wall of an example of a first forming tool, illustrating features of a fluid passage port.

FIG. 4C is a schematic magnified vertical section view of a portion of a wall of an example of a first forming tool, illustrating features of a fluid passage port.

FIG. 5 is a schematic longitudinal section view of an example of a first forming tool as an agglomeration of fibers is collecting on a forming surface thereof.

FIG. 6 is a schematic longitudinal section view of an example of a second forming tool.

FIG. 7 is a schematic longitudinal section view of the forming tools of FIGS. 2 and 6, shown being urged together to compress an agglomeration of fibers.

FIG. 8 is a schematic longitudinal section view of an example of a first formation of fibers.

FIG. 9 is a schematic longitudinal section view of an example of a third forming tool.

FIG. 10 is a schematic longitudinal section view of an example of a fourth forming tool.

FIG. 11 is a schematic longitudinal section view of the first formation of fibers of FIG. 8, disposed onto the third forming tool of FIG. 9.

FIG. 12 is a schematic longitudinal section view of the forming tools of FIGS. 9 and 10, shown being urged together to compress and change a formation angle of the first formation of fibers of FIG. 8.

FIG. 13 is a schematic longitudinal section view of an example of a second formation of fibers.

FIG. 14 is a schematic longitudinal section view of another example of a third forming tool.

FIG. 15 is a schematic longitudinal section view of another example of a fourth forming tool.

FIG. 16 is a schematic longitudinal section view of the first formation of fibers of FIG. 8, disposed onto the third forming tool of FIG. 14.

FIG. 17 is a schematic longitudinal section view of the forming tools of FIGS. 14 and 16, shown being urged together to compress, change a formation angle, and further reshape the first formation of fibers of FIG. 8.

FIG. 18 is a schematic longitudinal section view of another example of a second formation of fibers.

FIG. 19 is a schematic longitudinal section view of an example of a tampon applicator barrel blank.

FIG. 20 is a schematic longitudinal section view of the tampon applicator barrel blank of FIG. 16, converted to a tampon applicator barrel by creation of an ejection plunger passageway and petal profile cuts.

FIG. 21 is a schematic longitudinal section view of an example of tampon assembly including a tampon applicator barrel with formed petals, an ejection plunger, and an included compressed tampon pledget and withdrawal cord.

FIG. 22A is a perspective view of a non-limiting example of a forming surface shape.

FIG. 22B is a side elevation view of the shape shown in FIG. 22A.

FIG. 23A is a perspective view of another non-limiting example of a forming surface shape.

FIG. 23B is a side elevation view of the shape shown in FIG. 23A.

FIG. 23C is a longitudinal cross section of a non-limiting example of a forming surface shape.

FIG. 23D is a longitudinal cross section of a non-limiting example of a forming surface shape.

FIG. 23E is a depiction of a variety of non-limiting examples of base shapes.

FIG. 24 is a schematic section view of a portion of a non-limiting example of a first forming tool having a plurality of individual forming portions, having forming surface shapes.

FIG. 25 is a schematic perspective view of a tampon applicator barrel blank.

FIG. 26 is a black and white reproduction of a photograph of a prototype tampon applicator barrel blank, shown resting on a mobile phone over the activated camera light thereof, whereby the blank is illuminated from the inside.

### DETAILED DESCRIPTION

### Definitions

“Negative pressure” or “vacuum” (herein, used interchangeably) means fluid pressure proximate a downstream side of a fluid-permeable structure that is less than fluid pressure proximate an upstream side of the fluid-permeable structure, that tends to cause fluid to pass through the fluid permeable structure from the upstream side to the downstream side.

“Positive pressure” means fluid pressure proximate an upstream side of a fluid-permeable structure that is greater than fluid pressure proximate a downstream side of the fluid-permeable structure, that tends to cause fluid to pass through the fluid permeable structure from the upstream side to the downstream side.

### DESCRIPTION OF EXAMPLES

### Slurry

An example of a slurry suitable for use herein may include a carrier fluid such as water, and a suitable weight fraction of cellulosic pulp fibers, and if desired for particular examples, a fraction of non-cellulose fibers, and possibly one or more additives.

Depending upon the shape of the formed object and other factors including but not limited to color, mechanical strength, imperviousness or resistance to penetration by water or effects of humidity, and surface texture or finish required thereof, cellulosic fibers used to formulate the slurry may be selected from among the following non-limiting examples, including fibers obtained or derived from plants, such as wood fiber, wood pulp, and other natural plant fibers, and/or fibers spun from regenerated cellulose such as rayon, lyocell, viscose or cuprammonium rayon. Useful fibers may also include chemically treated natural plant fibers, such as mercerized pulps, chemically stiffened or crosslinked fibers, chemically oxidized fibers or sulfonated fibers. Useful fibers also may include are mercerized natural plant fibers, regenerated natural cellulosic fibers, fibers of cellulose produced by microbes, the rayon process, cellulose dissolution and coagulation spinning processes, and other cellulosic material or cellulosic derivatives. Other cellulosic fibers that may be useful include paper broke or recycled fibers and high yield fibers. High yield pulp fibers are those fibers produced by pulping processes providing a yield of about 65% or greater, more specifically about 75% or greater, and still more specifically about 75% to about 95%. Yield is the resulting amount of processed fibers expressed as a percentage of the initial wood mass. Such pulping processes include bleached chemithermomechanical pulp (BCTMP), chemithermomechanical pulp (CTMP), pressure/pressure thermomechanical pulp (PTMP), thermomechanical pulp (TMP), thermomechanical chemical pulp (TMCP), high yield sulfite pulps, and high yield Kraft pulps, all of which leave the resulting fibers with high levels of lignin but are still considered to be natural fibers.

Natural plant fibers that may be useful include cellulosic fibers obtained from plants, including wood fibers and wood pulp fibers, and non-wood cellulosic fibers. Non-limiting examples of hardwood fibers include fibers derived from hardwood sources such as eucalyptus, maple, birch, beech, oak, sweetgum, and aspen. Non-limiting examples of softwood fibers include fibers derived from softwood sources such as pine, spruce, and fir. Non-limiting examples of non-wood fibers include fibers derived from non-wood sources such as bamboo, cotton, abaca, kenaf, sabai grass, flax, esparto grass, straw, jute hemp, bagasse, milkweed floss, corn stover, miscanthus, pineapple leaf, and linen.

Semi-synthetic cellulosic fibers that may be useful include fibers spun from regenerated cellulose, such as viscose rayon, Lyocell and regenerated bamboo. Non-limiting examples of reclaimed/recycled cellulosic fibers include fibers derived from post-industrial recycled (PIR) waster or post-consumer recycled waste (PCR) and may include mixed paper, old newspaper/newsprint (ONP), old, corrugated containers (OCC), pulp substitutes (unprinted, uncoated, unadulterated paper and board), high-grade deinked, textile fibers.

Cellulosic pulp fibers can be prepared in high-yield or low-yield forms and can be pulped in any known method, including kraft, sulfite, high-yield pulping methods and other known pulping methods. Natural plant fibers contemplated by the present disclosure may include recycled fibers, virgin fibers or mixes thereof.

Non-limiting examples of chemically processed cellulosic fibers that may be useful include Kraft, sulfite, and soda fibers. Non-limiting examples of mechanically processed, refined fibers that may be useful include stone-ground wood, refiner-mechanical pulp (RMP), and thermomechanical pulp (TMP). Non-limiting examples of chemi-mechanically processed, refined fibers that may be useful include Chemiground wood, Cold soda, NSSC (Neutral sulfite semi-chemical), High yield sulfite, High yield kraft. Refined fibers typically have extending microfibrils, which provide for enhanced entanglement and additional fiber surface area for fiber-to-fiber hydrogen bonding, thereby providing added mechanical robustness to a formed structure, per unit weight of fibers included.

Generally, inclusion of long fibers may be preferred where mechanical strength of the formed object is a primary concern. Inclusion of short fibers may be preferred where a fine-grained and/or smooth surface appearance and feel and/or high opacity are primary concerns. In some examples, one may choose a blend of both long and short fibers, to strike a balance between the advantages and shortcomings of each. Herein, a short fiber is one having an average length less than 1.0 mm (Average Short Fiber Length-ASFL); a long fiber is one having an average length greater than 1.0 mm, from about 1.2 mm to about 3.5 mm, or from about 3 mm to about 10 mm (Average Long Fiber Length-ALFL). Where non-wood fibers are used, they may be in the long fiber range of length. For instance, bamboo can have a length from 1.1 to 2.0 mm and sunn hemp is even longer, it can have a length from 2.8 to 3.0 mm and sisal hemp can have a length from 2.5 to 2.7 mm. Kenaf can have a length from 2.7 to 3.0 mm, abaca can have a length from 4.0 to 4.3 mm.

In some examples, the slurry may include a minor fraction of fibers formed of thermoplastic polymeric, or synthetic, material(s). For example, thermoplastic polymeric fibers may be included: to provide a binding or stabilizing function (resulting from their entanglement among the cellulosic fibers, and plastic/melt deformation with application and heat and pressure to the molded object during its formation); to enhance surface finish; to add or improve resilience, flexibility, toughness and/or mechanical robustness to the molded object; to enhance resistance of the molded object to penetration by moisture, or to swelling that might result therefrom, etc.

The thermoplastic polymeric fibers or synthetic fibers may be formed of any suitable material(s), such as, but not limited to, material(s) selected from the group consisting of polyesters, polypropylenes, polyethylenes, polyethers, polyamides, polyhydroxyalkanoates, polysaccharides, and combinations thereof. The thermoplastic polymeric fibers or synthetic fibers may comprise a polymer. More specifically, the polymer material(s) forming fibers may be selected from the group consisting of poly(ethylene terephthalate), poly(butylene terephthalate), poly(1,4-cyclohexylenedimethylene terephthalate), isophthalic acid copolymers (e.g., terephthalate cyclohexylene-dimethylene isophthalate copolymer), ethylene glycol copolymers (e.g., ethylene terephthalate cyclohexylene-dimethylene copolymer), polycaprolactone, poly(hydroxyl ether ester), poly(hydroxyl ether amide), polyesteramide, poly(lactic acid), polyhydroxybutyrate, and combinations thereof. The polymer material(s) may comprise a segment, such as a polymer segment that may be complementary to a hydrophilizing agent and/or a segment thereof. The portion of the polymer segment that may be complementary to a hydrophilizing agent may facilitate association between the synthetic fiber and the hydrophilizing agent. The complementary segment may comprise a polyester segment. The polyester segment may further comprise a polyethylene terephthalate segment. The complementary segment of the polymer may be located on the surface of the synthetic fiber. Such may be the situation wherein the synthetic fiber may be a bicomponent fiber comprising a core and an outer surface.

The thermoplastic polymeric fibers or synthetic fibers may be a single component (i.e., single synthetic material or mixture makes up entire fiber), or bicomponent or multi-component (i.e., the fiber in cross section is divided into regions of differing composition. Bicomponent fibers may be of a sheath-core configuration, a side-by-side configuration, or any other desired configuration. In some examples, bicomponent fibers having a polymer sheath component having a first melting temperature, and a core component of a second melting temperature higher than the first melting temperature, may be included. In a more specific examples, a sheath component me comprise a polyethylene surrounding a core component of a polymer having a greater melting temperature, such that the polyethylene sheath component may plastically deform, soften, melt and/or fuse and/or bond with other fibers, while the core component remains solid, in response to heating of the object being molded, during its formation.

Fibers formed of synthetic polymer component(s) may be included as component fibers, or they may be included to provide a binding material for the other (e.g., cellulosic) fibers included to form the molded object. Any or all of the synthetic fibers included may be treated before, during, or after the process of the present invention to change any desired properties of the fibers. For example, in certain embodiments, it may be desirable to treat the synthetic fibers before or during the forming process described herein, to make them more hydrophilic or wettable to enhance their dispersion within the slurry.

In examples of a tampon applicator barrel specifically described herein, the slurry may be constituted of water and a weight fraction of 0.2 percent to 3 percent cellulosic fibers. A majority weight fraction of the cellulosic fibers is preferably long fibers, for example, northern softwood kraft (NSK) or southern softwood kraft (SSK) fibers, which are typically long fibers, or a blend thereof. In some examples, the majority weight fraction of the cellulosic fibers is preferably equal to or greater than 90 percent NSK and/or SSK fibers, or substantially entirely NSK and/or SSK fibers.

“Additive” as used herein refers to a non-cellulosic material included with the cellulosic material (i.e., different fiber types) in the slurry. Additives are distinguished from coating in that coatings are applied to one or more surfaces of the article after it is formed, while additives are included in the slurry and therefore generally incorporated within and evenly dispersed throughout the cellulosic material forming a region of the article.

Additives may impart any of a number of properties to the cellulosic material and therefore to the region of the article. These properties may derive primarily from the incorporation of the additive itself or may further be modified by any post-formation treatment of the article such as drying or compressing.

Additives may be used for “sizing” or to impart resistance to fluid penetration to the cellulosic material. Non-limiting examples of sizing agents include non-reactive systems such as resin systems commonly used in acidic papermaking; natural resin can be processed from southern pines tall oil (a byproduct from alkaline pulping); and reactive systems such as those based on alkyl ketone dimers or alkenylsuccinic anhydrides. Other non-limiting examples of sizing agents may include acid chlorides, acid anhydrides, enol esters, alkyl isocyanates, rosin anhydrides, starch (including amylose and/or amylopectin), animal glue, methyl cellulose, carboxymethyl cellulose (CMC), polyvinyl alcohol, waxes and wax emulsions, alginates, or polymers such as Styrene Maleic Anhydride (SMA), polyurethanes, and styrene acrylate.

Additives may be used to provide strength to the cellulosic material including wet strength and/or dry strength. Non-limiting examples of wet strength additives include thermosetting resins such as amino resins including urea-formaldehyde resins, thermosetting aminoplastic resins such as melamine formaldehyde resins, amine-epichlorohydrin resins such as amine-epichlorohydrin polymeric resins, polyamide-epichlorohydrin resins, polyamide-amine-epichlorohydrin (PAE) resins and the like, glyoxalated polyacrylamide (GPAM) resins including crosslinked and non-crosslinked GPAM polymers, modified starch such as dialdehyde starch, and/or polycarboxylic acids. Non-limiting examples of dry strength additives include polysaccharides and modified polysaccharides such as starch (including amylose and/or amylopectin), modified starch, modified cellulose(s) such as carboxymethyl cellulose (CMC) and/or chitosan, as well as crosslinked/branched polysaccharides such as hemicellulose(s) and/or polysaccharide gums including locust gum and/or tamarind gum and/or guar gum among others, cationic and/or anionic modified polysaccharides including combinations of cationic and anionic modified polysaccharides as polyelectrolyte multilayers (PEM's), polyacrylamides (PAMs), and latex additives.

Additives may also include colorants (i.e., dyes and pigments). It has been found that fiber-based articles may be relatively unaffected in quality, by addition of limited quantities of pigments, colorants and filler materials, provided that the pigment material selected is not chemically reactive with water or any of the other components of the molding composite under the molding conditions referenced herein. Accordingly, the composite described herein enables the manufacturer to mold parts in a large variety of colors. In order to avoid substantial effect on the moldability of the composite, it is believed that pigment, if desired, should be included to a maximum of 5 percent, more preferably 4 percent, and still more preferably 3 percent, by weight of the dry component blend.

Additives may also include fillers which impart various properties to the article or article region including brightness, opacity, whiteness, gloss, smoothness, and printability. Fillers can also be used to modify the density of the article or region. Non-limiting examples of fillers include titanium dioxide, clays such as kaolin, calcium carbonate, aluminum trihydrate, silicas, silicates and aluminosilicates, and calcium sulfoaluminates.

Additives may include binders that impart structural integrity to the article. Any suitable binder may be used, but binders that exhibit human bio-compatibility, relatively rapid biodegradability, source sustainability and dispersibility are preferred. Polysaccharides including starches (e.g., corn starch and/or potato starch) may be suitable binders.

Additives may include dispersing agents that may facilitate the dispersion of a co-additive in the slurry. Dispersing agents may be particularly useful for dispersing co-additives which are not fully water-soluble such as hydrophobic or hydrophobically-modified additives, or particulate additives. Non-limiting examples of dispersing agents include salts of carboxymethylcellulose such as sodium carboxymethylcellulose (CMC salt).

Additives may include lubricating agents that may improve releasability of the molded article from the mold (i.e., reducing chances that the molded object will stick to the mold). Non-limiting examples of lubricating agents may include long-chain fatty acids and salts thereof such as non-alkali metal salts thereof such as calcium stearate, magnesium stearate, zinc stearate, calcium laurate, magnesium laurate, zinc laurate, aluminum laurate, strontium laurate, aluminum stearate, strontium stearate, and mixtures thereof.

If a lubricating agent is desired the lubricating agent may preferably be included in a quantity from 0.2 to 2.0 weight percent of the total dry component blend.

Additives may be at least partially water-soluble and may be at least partially dissolved in the slurry or may be particulate. The additive(s) may be suspended in the slurry as particles or as emulsions or dispersions.

### Equipment Configuration and Process

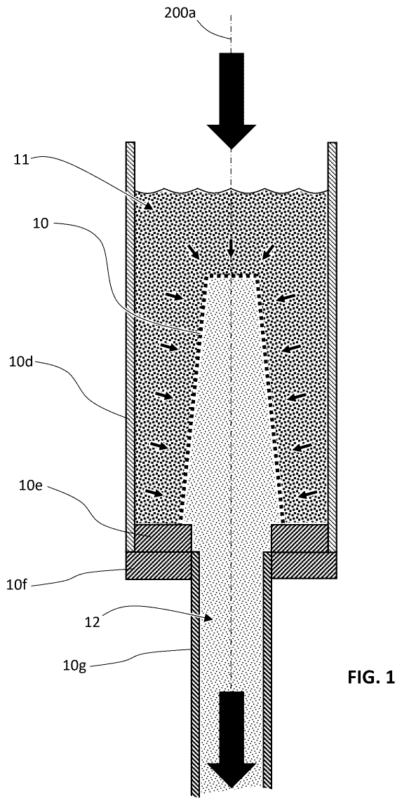

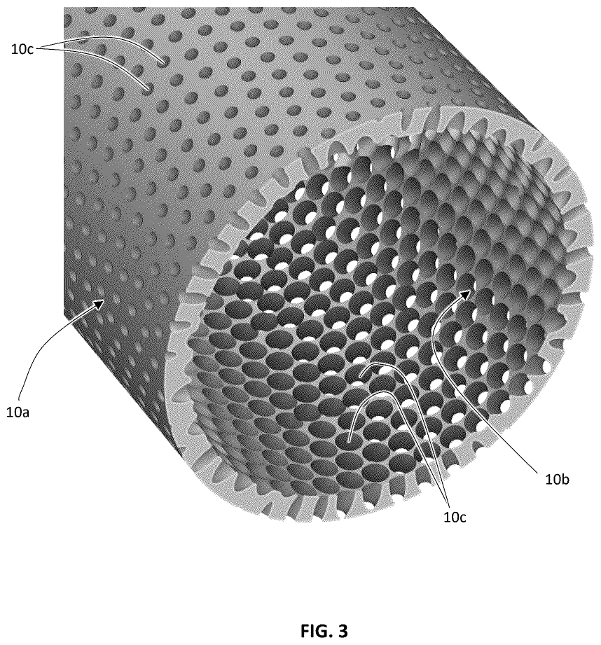

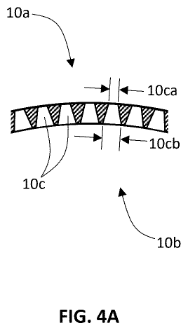

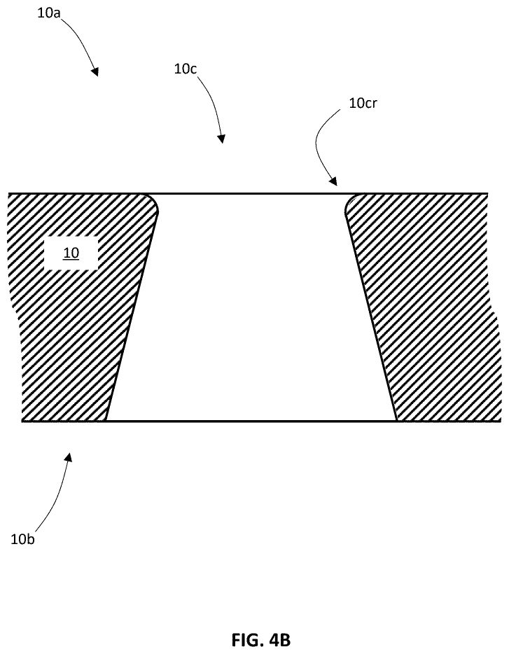

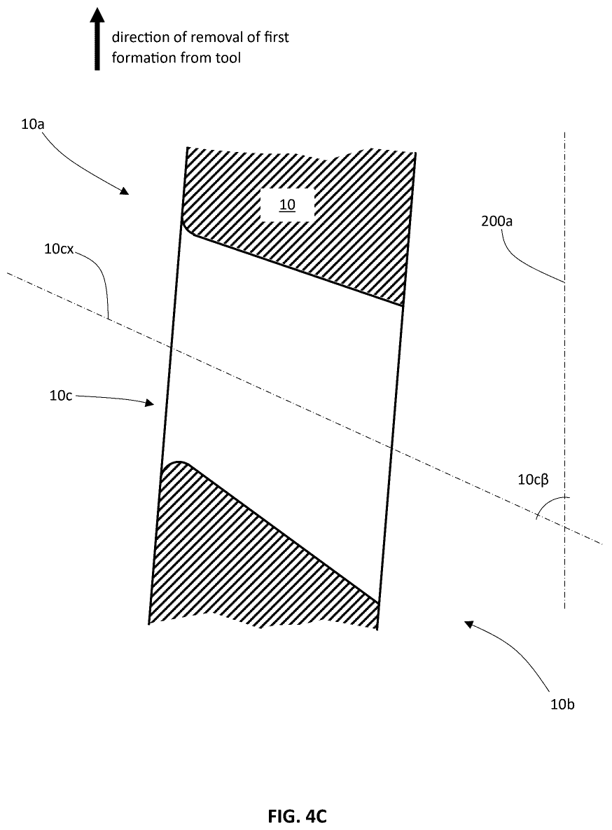

Referring to FIGS. 1-4C, a first forming tool 10 is a porous structure having a first forming surface 10a, a first non-forming surface 10b, and a pattern of a plurality of fluid passage ports 10c, which are ports extending from first forming surface 10a to first non-forming surface 10b. As may be appreciated from FIGS. 3 and 4, the fluid passage ports 10c may be formed into the tool structure such that the sizes of their openings 10cb at the first non-forming surface 10b are larger than the sizes of their openings 10ca at the first forming surface 10a. Where ports 10c are circular in cross section, the size of an opening at the surface is the diameter of the port at the surface. Where ports 10c have a cross sectional shape other than circular, the size of an opening at the surface is the largest dimension of the port at the surface. Preferably, ports 10c have a rounded cross-sectional shape that has no sharp inside corners, such as but not limited to oval, ovoid, elliptical, stadium, or circular. Preferably, the shape is circular.

As the process is described further below, the cross-section shape and opening size differential of the ports 10c between the first forming surface 10a and first non-forming surface 10b may help ensure that fibers that may incidentally pass through ports 10c during the agglomeration step do not catch in, and potentially clog them.

Additionally, the edges 10cr defining fluid passage ports 10c may be rounded or radiused at the first forming surface 10a, as suggested in FIG. 4B; this may help facilitate removal of a first formation from first forming tool 10, as described below. Referring to FIG. 4C, in some examples, for portions of a first forming tool that have a first forming angle α1 as described below, a plurality, majority or substantially all of ports 10C on such portions may be formed such that their center axes 10cx form removal facilitating angles 10cß with respect to a longitudinal axis 200a of the first forming tool. Such configuration may help ease removal of a first formation 101 from the forming tool 10, in the process described below, by reducing the resistance to removal that may be caused by fibers of the first formation 101 extending therefrom into ports 10c, following formation, and thereby potentially obstructing removal. If this feature is included, the ports should be configured such that their center axes 10cx are angled, with respect to the longitudinal axis 200a, toward the direction of removal of the first formation from the tool (as reflected in FIG. 4C). Preferably, removal facilitating angles 10cß are in a range of 15 degrees to 75 degrees, preferably 25 degrees to 65 degrees, and more preferably 35 degrees to 55 degrees.

In some circumstances, potential for catching of fibers within ports 10c may be less of a concern. In such circumstances, it may be desirable that ports 10c have a cross-section shape may be better suited or a more closely-arranged pattern, or nesting, of port shapes. In such circumstances, it might be desired to include ports having polyhedral or rounded-polyhedral shapes, such as triangle, square, rectangle, pentagonal, hexagonal, or other polyhedral shapes. Hexagonal shapes may be deemed useful for their suitability for being closely arranged (e.g., in a honeycomb pattern), combined with the relatively large size of their corner angles (120 degrees), making them less likely to catch fibers that may be drawn in to the ports 10c, than shapes having fewer sides.

The size of the openings 10ca at the first forming surface 10a may be selected in view of the size of the fibers to constitute the slurry, to strike a balance between the competing objectives of ease and efficiency of pumping or drawing carrier fluid through the first forming tool 10 (i.e., maximizing speed and minimizing pump energy required), and effectively trapping fibers and accumulating fibers on the first forming surface 10a, rather than allowing them to pass through the tool. In particular examples in which NSK fibers (which are typically long fibers) may be selected as the predominant fiber component to form the desired structure the size of the openings 10ca may be 0.5 mm to 1.2 mm, preferably 0.6 mm to 1.0 mm. In such examples, it may be desired that the openings be substantially circular in cross section, which can help minimize the potential for fibers to become lodged in the openings, clog them, and thereby adversely impact the ability of the tool to agglomerate an even distribution of fibers on the forming surface 10a.

The pattern of fluid passage ports 10c may be arranged to effect a relatively evenly distributed accumulation of fibers on the first forming surface 10a for a relatively uniform basis weight and/or caliper of the formed structure. In such examples, the numerical density of fluid passage ports 10c, i.e., the number of fluid passage ports per unit surface area of the first forming surface 10a, may be substantially consistent along the majority or substantial entirety of the first forming surface. In such examples, the sizes of the fluid passage ports and their openings at the first forming surface 10a and first non-forming surface 10b may be substantially consistent throughout the pattern.

Alternatively, the pattern of fluid passage ports 10c may be arranged to effect a distribution/accumulation of fibers that varies along the forming surface 10a, for a varying basis weight and/or caliper of the formed structure. In such examples, the numerical density of fluid passage ports 10c, i.e., the number of fluid passage ports per unit surface area of the first forming surface 10a, may be varied along the first forming surface. In such examples, the sizes of the fluid passage ports and their openings at the first forming surface 10a and first non-forming surface 10b may be substantially consistent throughout the pattern. Alternatively, or additionally, the sizes of the fluid passage ports and their openings at the first forming surface 10a may also be varied. Smaller ports will allow carrier fluid to pass more slowly, and thereby provide less accumulation of fiber on the associated portion of the forming surface; and larger ports will allow carrier fluid to pass more rapidly, and thereby provide greater accumulation of fibers on the associated portion of the forming surface. For the particular non-limiting example of formation of a tampon applicator barrel described herein, it may be desired that the grip (rearward) end of the barrel be stiffer and stronger, and that the petal (forward) end of the barrel be more flexible. Accordingly, the first forming tool 10 and forming surface 10a thereof may have a greater numerical density of fluid passage ports 10c and/or larger openings at the portion of forming surface 10a that will form the grip end, and a lesser numerical density of fluid passage ports 10c and/or smaller openings at the portion of forming surface 10a that will form the petal end.

As contemplated herein, tooling such as first forming tool 10 may be configured to form a 3D structure having respective facing surfaces, or features with respective facing surfaces, that lie at acute angles, at relatively small values, with respect to each other. As explained in the background, as surfaces of a desired structure to be formed approach a parallel configuration, a simple process for formation thereof becomes difficult. Accordingly, tooling such as first forming tool 10 may have a first forming surface 10a with facing portions that are not parallel, but rather, lie at acute angles, at relatively small values, with respect to each other.

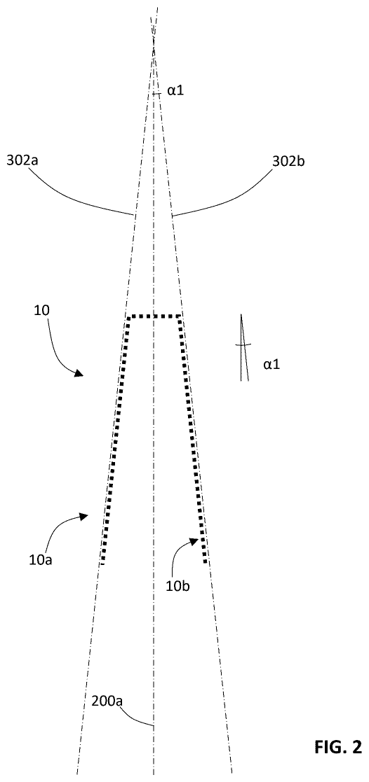

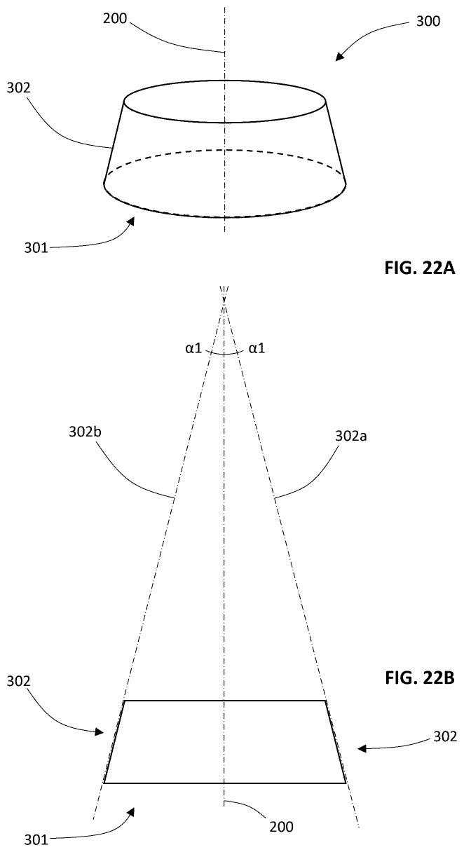

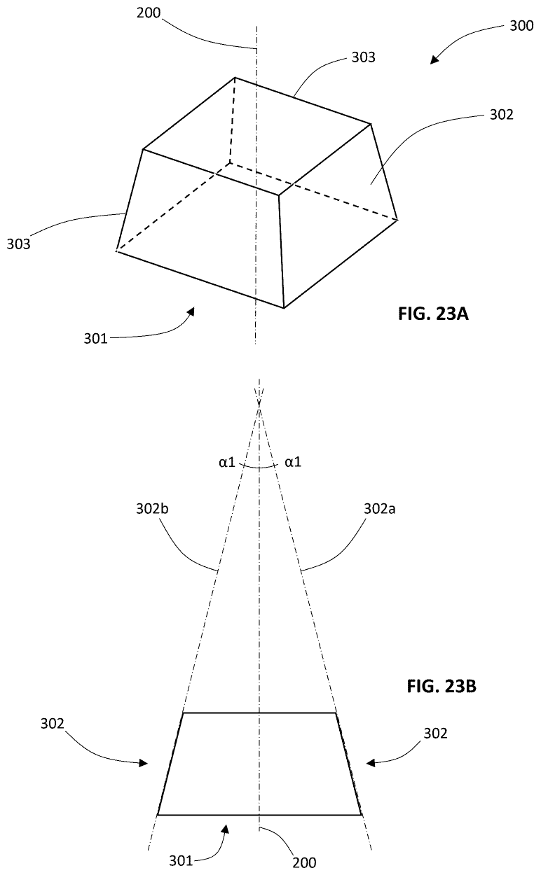

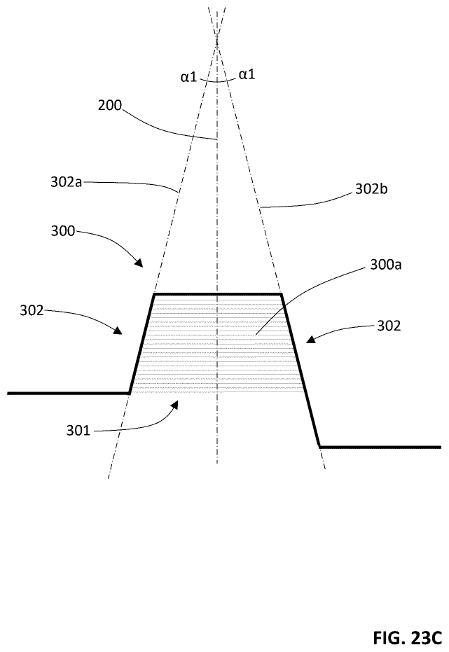

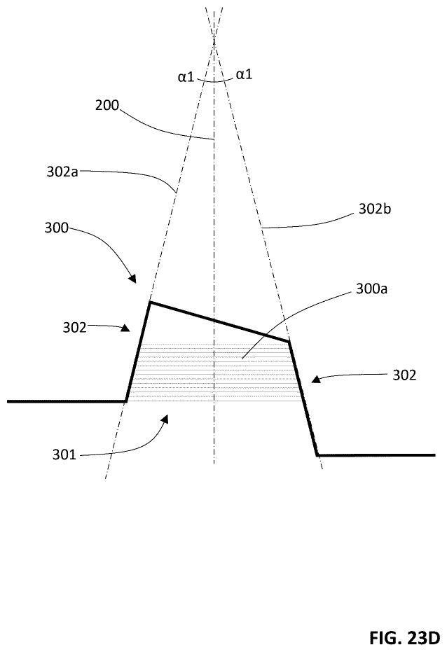

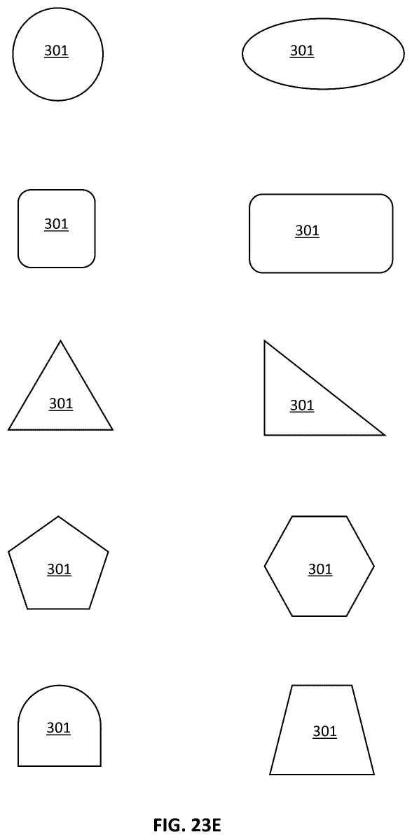

In the non-limiting example of a first forming tool depicted in FIGS. 1 and 2, the 3D shape of the first forming surface 10a is contemplated to be that of a right circular conical frustum, having a circular 2D base shape, and being configured to form a first agglomeration of fibers generally having a similar 3D shape. In other examples, a first forming surface of a first forming tool may have a 3D shape 300 with a 2D base shape 301 that is oval, ovoid, elliptical, stadium-shape, triangular, square, rectangular or other polyhedron shape. Referring to additional non-limiting examples of forming surface shapes 300 depicted in FIGS. 22A, 22b, 23A and 23B, a 3D forming surface shape has a 2D base shape 301 and one or more sides 302. Side portions 302 of first forming surface 10a occupy at least two identifiable converging planes 302a, 302b that form a first forming angle α1 with a longitudinal axis 200. In the case of a pyramid or pyramidal frustum, walls or side portions 302 may be defined by edges 303. Edges 303 may be sharply defined, or rounded. If the 3D forming surface shape has a base shape that is circular, other rounded, shape, or polyhedral, the 3D forming surface shape will have a longitudinal axis 200, which is perpendicular to the plane occupied by the 2D base shape 301, and geometrically centered therein. It may be appreciated that in some circumstances a base shape 301 of a 3D forming surface shape 300 may not be readily apparent, such as where, for example, a forming surface is configured to form an object or feature thereof having varying depths, levels, elevations, etc. To illustrate, referring to FIGS. 23C and 23D, where a base shape is not quickly identifiable, an alternative identifying characteristic of a longitudinal axis 200 is that converging planes 302a, 302b, along lines occupied by portions of the 3D forming surface side portions 302, form symmetric angles with respect to an identifiable longitudinal axis 200, in at least one third plane. The side portions 302 in each figure, lie along planes 302a, 302b that are converging and facing across the region 300a shown shaded, and symmetrically-angled with respect to a longitudinal axis 200 in the plane of the page. In FIGS. 23C and 23D, forming surface shapes 300 have respective side portions 302 of differing dimensions (with respect to the figures as shown on the pages, differing heights). In the planes of the pages, however, longitudinal axes 200 may be identified, about which converging planes 302a, 302b extending along sides 302 form symmetric (equal) angles α1. This will be true if the 3D shape has any portion that is conical or pyramidal, in part or in whole. It will be appreciated that the cross sections shown in FIGS. 23C and 23D could be profiles of any forming shape having a base shape 301 that can be identified at the bottom of the regions 300a shown shaded, i.e., base shapes associated with these cross section profiles could be circular, oval, ovoid, elliptical, triangular, square, rectangular or any other polyhedral shape, or even combinations of shapes. Non-limiting examples of other possible base shapes 301 are illustrated in FIG. 23E.

If no such longitudinal axis 200 can be identified, then the 3D shape is not configured for a fiber formation that will have side portions that approach being parallel with each other, and is not within the scope of contemplation herein. For example, a forming surface that is entirely hemispherical has no planar or linear portions, and therefore, is not within the scope of contemplation herein. On the other hand, a compound shape including a frustoconical portion merging into a hemispherical portion at its narrower end, will be within the scope of contemplation herein, because a longitudinal axis may be identified as described, about which surfaces of the frustoconical portion lie along converging planes that are symmetrically angled about such an axis.

It will be appreciated that formation, or object or feature formed as described herein, will reflect the geometry of the tooling used to form it, and therefore, will have an identifiable longitudinal axis and formation angles, or not, according to the discussion above.

In the non-limiting example depicted in FIG. 2, first longitudinal axis 200a is identified. Since forming very shallow features having parallel facing surfaces into a structure is less likely to face the challenges described herein, first forming surface 10a of first forming tool 10 may generally have a longitudinal dimension equal to or greater than 5 mm; and addition, the first forming surface may generally have an aspect ratio of longitudinal dimension to greatest lateral dimension of the particular structure or feature thereof, of at least 1.5, up to 6. The resulting formation(s) 101, 102 and resulting formed object, therefore, may have a similar aspect ratio.

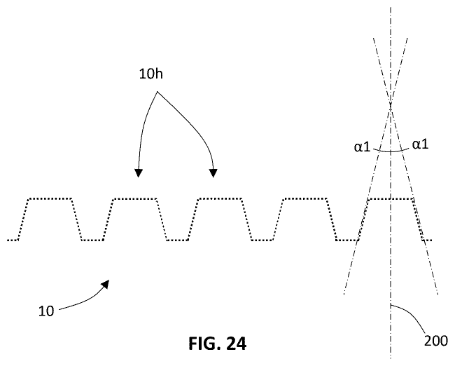

As may be appreciated from FIG. 24, it is contemplated herein that a first forming tool may include or form a portion of a forming surface that includes a plurality of individual portions 10h each having 3D shapes as described herein. The 3D shapes of the individual portions may all be of similar size and shape, or they may vary in one or both of size and shape. In one non-limiting example, the shapes may be adapted to form individual compartments or pockets in a formed structure configured to be a blister package component.

It is also contemplated that, depending upon the structure to be formed and the desired shape and features thereof, the respective first, second, third, fourth, etc., forming tools to be used in the process steps may be configured as male, female, male, female, etc., respectively, or alternatively, female, male, female, male, etc., respectively. This means that, for example, during the forming process, a first male forming tool has outward-facing surfaces onto which fiber is first agglomerated, and following fiber agglomeration is fitted inside a second forming tool, etc., or alternatively, a first female forming tool has inward-facing surfaces onto which fiber is agglomerated, and following fiber agglomeration is fitted to the outside of a second male forming tool, etc. In the non-limiting examples for forming a tampon applicator barrel depicted in FIGS. 1-17, the depicted first, second, third and fourth forming tools 10, 20, 30, 40 might be characterized as male, female, male, female, respectively. Both forming configurations and sequences are contemplated herein.

A first forming tool may be manufactured to include a first forming angle α1 of 1.5 to 5 degrees. The first forming angle is the angle that is formed between a shaped forming surface and the longitudinal axis of the shape, along a longitudinal cross section taken through a plane occupied by the longitudinal axis. In the example shown in FIG. 2, first forming angle α1 between first forming surface 10a and longitudinal axis 200a is identified.

As reflected in FIG. 1, the base of active portion of first forming tool 10 may be mounted to a base flange 10e or similar supporting component, by any suitable means. During the early steps of the process described below, first forming tool 10 with base flange 10e may be removably but sealingly, directly or indirectly, affixed to a drain flange 10f or similar fixture, which in turn may be sealingly affixed to, or be integral with, a drain pipe 10g. A slurry vessel 10d may be removably but sealingly affixed about base flange 10e and/or drain flange 10f.

Drain pipe 10g may be connected directly or indirectly to a slurry carrier fluid receiving element (not shown). A vacuum/negative pressure pump (not shown) may be disposed in line with drain pipe 10g, and configured to draw slurry carrier fluid through the fluid passage ports of first forming tool 10. Alternatively or additionally, a positive pressure pump (not shown) may be disposed upstream of slurry vessel 10d and configured to pressurize slurry to urge it against first forming tool 10, and thereby urge carrier fluid through the fluid passage ports of first forming tool 10.

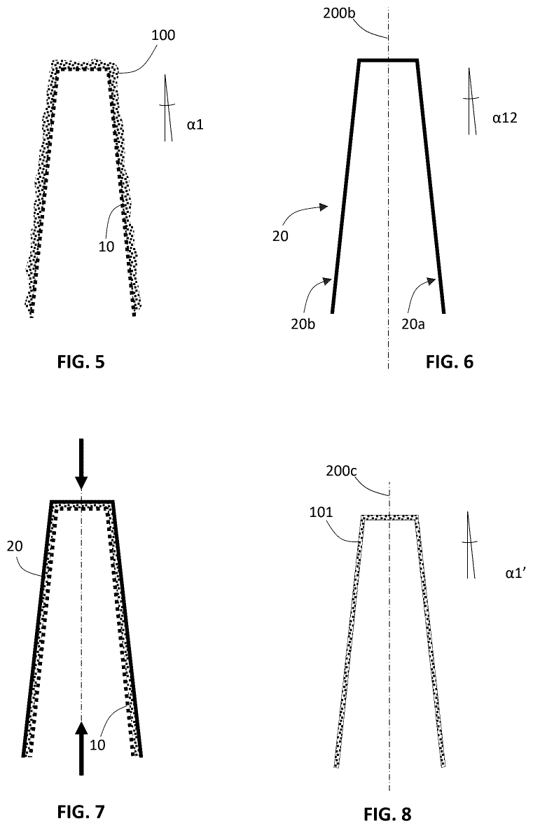

Referring now to FIGS. 1, 2 and 5, following preparation of a slurry of the desired composition of carrier fluid (e.g., water) and cellulosic fibers, a measured quantity of the slurry 11 sufficient to contain the desired weight quantity of fibers, to manufacture the desired structure, is introduced into the slurry vessel 10d. The slurry is then forced via positive pressure, or drawn via vacuum/negative pressure, against the first forming surface 10a of first forming tool 10, and carrier fluid 12 is urged or drawn through fluid passage ports 10c, in the general manner and direction indicated by the arrows in FIGS. 1 and 5. During this step, the first forming tool filters a majority of the suspended fibers from the carrier fluid, and they collect and agglomerate on the first forming surface 10a, to form fiber agglomeration 100 thereon. If needed, agitating equipment (not shown) may be configured to agitate the slurry within the slurry vessel 10d during this agglomerating step, to reduce fiber settling and help maintain substantially even dispersion of fibers in the slurry as it is being drawn or pumped through first forming tool 10, and thereby help promote even agglomeration of fibers on first forming surface 10a. This measure may be particularly desired in examples in which the first forming surface has a large surface area, or where the geometry of the first forming surface is more complex, e.g., includes a plurality of 3D features to be imparted to the desired structure, for example, with a first forming tool configured to produce a blister package component with a plurality of pockets or compartments.

As suggested in FIG. 1, it may be desired to pump and/or draw the slurry through the first forming tool in a nearly or substantially vertical direction, wherein the longitudinal axis 200a of the first forming tool (e.g., first forming tool 10) is oriented vertically. This can also help promote even agglomeration of fibers along horizontal planes through the first forming tool, by mitigating settling effects of gravity on fiber dispersion/distribution in the slurry—i.e., help avoid greater accumulation of fibers on one side of features of the first forming tool, than on the other.

Additionally, the pattern of fluid passage ports 10c may be configured to compensate for the feature of the process whereby the level of liquidous slurry 11 being pumped or drawn through the tool falls below the uppermost features of the first forming tool first, and continues to remain in contact with the lowermost features for the longer duration of the agglomerating step—i.e., there is more time for the lowermost features of the first forming tool to agglomerate fibers, than for the uppermost features. Thus, in some examples, it may be desired to arrange and configure the pattern of fluid passage ports such that they have one or both of greater numerical density and greater size at the uppermost regions of the first forming surface, and one or both of lesser numerical density and smaller size at the lowermost regions of the first forming surface, with a transition between these features along the height of the first forming tool. This configuration can help promote even agglomeration of fibers from the upper portions to the lower portions of the first forming surface, as the slurry level falls as the measured quantity of slurry in the slurry vessel is being pumped or drawn through the first forming tool. In other alternative examples, however, it may be desired to provide a continuous supply of slurry to the slurry vessel, so that the level thereof does not fall below the uppermost features of the tool during the agglomeration step. The extent and basis weight of the agglomeration on the forming surface may then be controlled by regulating the time of operation of the pump and/or time of immersion of the first forming tool in the slurry while the pump is operating.

Referring to FIGS. 5-8, following the agglomeration step described above, a shaped fiber agglomeration 100 is present on the first forming surface of first forming tool 10. Prior to further processing, this fiber agglomeration 100 still has substantial carrier fluid content, is relatively soft and pasty and mechanically unstable, and cannot be easily removed from the first forming tool without deforming or damaging it. The next step is to stabilize the agglomeration, and remove a portion of carrier fluid from it.

A second forming tool 20 may be provided. Second forming tool 20 has a second forming surface 20a, a second non-forming surface 20b, and a longitudinal axis 200b. Second forming surface 20a may be sized and shaped to follow the shape and contours of the first forming surface 10a of first forming tool 10 and conformably fit thereabout, with a desired clearance therebetween, when the longitudinal axes 200a, 200b of the two tools are aligned and co-located. Second forming surface 20a may have a first adjusted forming angle α12 that is substantially close to or matches that of first forming surface 10a of first forming tool 10. Alternatively, if a variation in fiber densification, along the length or other feature of the structure being formed, is desired to be imparted in the step described below, second forming surface 20a may have a forming angle that deviates from the first forming angle α1. In some examples in which first forming angle α1 is relatively small, it may be desired that first adjusted forming angle α12 be slightly greater than first forming angle α1. This can help reduce displacement of fibers in the agglomeration via contact and shearing friction with second forming surface 20a, as second forming tool 20 is brought longitudinally into proximity with first forming tool 10 and compression of fiber agglomeration begins. For this purpose, in some examples, the difference between first forming angle α1 and second forming angle α12 may be 0.5 to 1.5 degrees.

Referring to FIGS. 5-8, first forming tool 10, bearing fiber agglomeration 100, may be removed from the slurry vessel and brought into alignment with second forming tool 20, and the two tools may be urged together (as suggested by the large arrows in FIG. 7) with their longitudinal axes aligned and co-located. As forming tools 10 and 20 are urged together in this manner, fiber agglomeration 100 is compressed between them. This consolidates and densifies the fibers, expresses liquid carrier fluid in from the agglomeration 100, and shapes the surface of the agglomeration that faces second forming surface 20a. It will be appreciated that the geometry of the forming angle(s) in the opposing first and second forming surfaces 10a, 20a enables compression of the agglomeration between forming surfaces 10a, 20a, along a compression direction (first forming direction) having a vector component normal to the forming surfaces. It will be appreciated, therefore, that the forming angle α1 should not be so small as to substantially eliminate this vector component, and thereby not effect compression by urging the tools together longitudinally as described. Further, if forming angle α1 is too small, agglomeration 100 is more likely to be substantially deformed or damaged by excessive displacement of fibers of the agglomeration, resulting from contact and shearing friction with second forming surface 20a. At the same time, however, toward the goal of forming a structure having facing parallel surfaces or sides (such as a structure having, e.g., a cylindrical, square or rectangular shape) as efficiently as possible, it may be desired that forming angle α1 be as small as feasible. To strike an appropriate balance, it may be desired to manufacture first forming tool 10 to have first forming surface 10a with first forming angle α1 of 3 degrees to 10 degrees.

Via such compression a substantial portion of carrier fluid remaining in the agglomeration 100, following its formation, may be expressed therefrom in liquid form, via pressure between the tools 10 and 20, through the fluid passage ports 10c, or through additional fluid exit ports (not shown) that may be provided in second forming tool 20. Additionally, or alternatively, one or both of forming tools 10, 20 may be provided with heating energy, whereby carrier fluid may be heated, vaporized and driven out of the agglomeration in steam/vapor form through the fluid passage ports 10c, and/or through additional fluid exit ports (not shown) that may be provided in second forming tool 20. In addition to vaporizing carrier fluid and driving it out of the structure, heat can help impart a set to the fibers and thereby add stiffness and strength to the structure.

With such consolidation/densification of fibers and removal of carrier fluid, fiber agglomeration 100 is converted to first formation 101. First formation 101 will have a longitudinal axis 200c that, prior to removal from first forming tool 10, is substantially aligned and co-located with axes 200a, 200b of the first and second forming tools 10, 20, while they remain in the compressing position. First formation 101 will be effectively structurally stable following this stage of compression (and if desired, heating), enabling removal of second forming tool 20, and enabling removal of first formation 101 from first forming tool 10, without substantial damage. As noted above, the edges 10cr defining the fluid passage ports 10c at first forming surface 10a, may be radiused or rounded (see, e.g., FIG. 4B), which can help ease removal of first formation 101 from first forming tool 10 by mitigating resistance to removal that may be caused by any stray fibers that may have been partially drawn into fluid passage ports 10c from the main portion of fiber agglomeration 100 during the agglomerating step described above. As a result of its formation by first and second forming tools 10 and 20, upon its removal from first forming tool 10, first formation 101 is imparted with a first formation angle α1′ that is similar or approximately equal to first forming angle α1. For the purpose of facilitating removal from the tools and further formation steps described below, it may be desired that carrier fluid removal is controlled so that first formation 101 retains a weight percentage of carrier fluid (e.g., water) of 25 percent to 60 percent, thereby leaving the first formation 101 still somewhat malleable and re-formable.

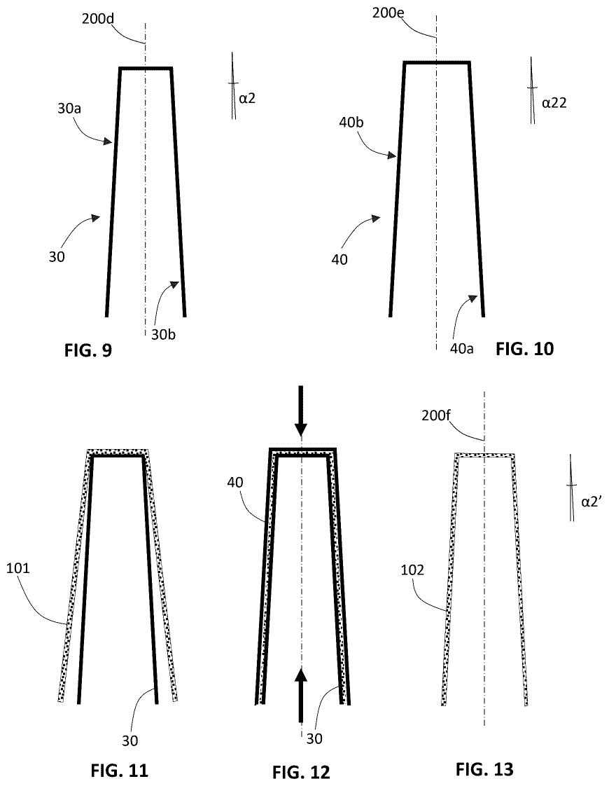

Referring now to FIGS. 9-13, a third forming tool 30 and a fourth forming tool 40 may be provided. Third forming tool 30 has a third forming surface 30a, a third non-forming surface 30b, and a longitudinal axis 200d. The third forming surface 30a may be imparted with a second forming angle α2 that is less than first forming angle α1 and/or first formation angle α1′, and otherwise be sized and shaped such that first formation 101 will fit thereover (or therewithin). Fourth forming tool 40 has a fourth forming surface 40a, a fourth non-forming surface 40b, and a longitudinal axis 200e. The fourth forming surface 40a may be imparted with an adjusted second forming angle α22 that is less than first forming angle α1 and/or first formation angle α1′, and may be similar or approximately equal to second forming angle α2; or may be slightly larger than second forming angle α2 within the range and for the purpose described above, with respect to first forming angle α1 and first adjusted forming angle α12. The fourth forming surface 40a may be imparted with a shape that follows the shape, size and contours of third forming surface 30a of third forming tool 30, and conformably fits thereabout, with a desired clearance therebetween, when the longitudinal axes 200d, 200e of the two tools are aligned and co-located and the tools are brought together as will be described.

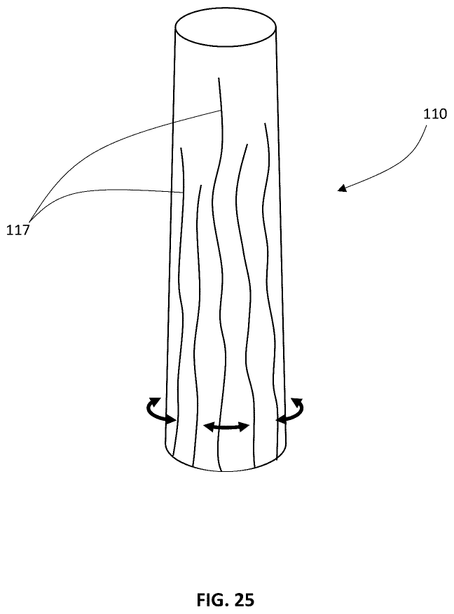

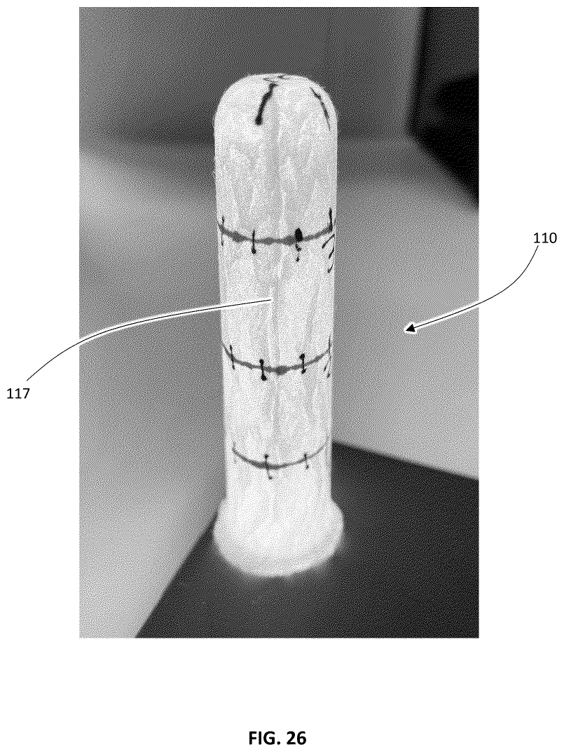

As a next step in progressive formation of the desired structure, first formation 101 may be placed onto (or into, as appropriate) third forming tool 30. Next, fourth forming tool 40 may be placed over (or inside, as appropriate) first formation 101 and third forming tool 30, with the longitudinal axes of forming tools 30, 40, substantially aligned and co-located. Next, forming tools 30, 40 may be urged together longitudinally to re-form, reshape, and further compress first formation 101, as suggested in FIG. 12. Because second forming angle α2 is less than first forming angle α1 and/or first formation angle α1′, portions of first formation 101 are compressed along a direction normal to the fourth forming surface 40a, and also laterally and perimeter-wise (and in the conical examples depicted, circumferentially/hoop-wise), such that first formation 101 is forced into a new configuration conforming in shape and size with third forming surface 30a and fourth forming surface 40a, thereby having a reduced, second formation angle α2′. In some examples, without intending to be bound by theory, it is believed that such lateral perimeter-wise compression induces creation of fine wrinkles 117 in portions of the final structure, particularly those most extensively compressed laterally/perimeter-wise and re-formed in this step, as indicated by the large curving arrows shown in FIG. 25. Wrinkles may be somewhat irregular and randomly-distributed about the final structure, but oriented generally in a longitudinal direction. See, e.g., FIGS. 25 and 26 (wrinkles 117, in tampon applicator barrel blank 110). It is believed that similar, generally longitudinally-oriented wrinkles will appear in structures of other shapes, progressively formed as described herein, as well.

Via such compression a second substantial portion of carrier fluid remaining in the first formation 101 may be expressed therefrom, via pressure between the tools 10 and 20. If desired, one or both of third forming tool 30 and fourth forming tool 40 may be provided with fluid exit ports (not shown). Alternatively, in some examples, it may be desired that one or both of third forming surface 30a and fourth forming surface 40a have fewer fluid exit ports than a previously-used or opposing forming surface, or none at all, to facilitate impressing and imparting smooth surface finishes to the inside and/or outside of the structure. One or both of forming tools 30, 40 may be provided with heating energy, whereby carrier fluid may be driven out of the first formation 101 in steam/vapor form through, either through gap(s) between edges of the tools where the formation remains exposed, or through fluid exit ports that may be provided. In addition to vaporizing a substantial portion of remaining fluid and driving it out of the structure, heat will help impart further set to the fibers and thereby add further stiffness and strength to the structure.

With such further compression and re-forming, first formation 101 is converted to second formation 102. Second formation 102 will have a longitudinal axis 200f that, prior to removal from third forming tool 30 and fourth forming tool 40, is substantially aligned and co-located with axes 200d, 200e of the third and fourth forming tools 30, 40, while they remain in the compressing position. Second formation 102 will be structurally more rigid following this stage of compression and re-formation (and if desired, heating), facilitating its removal from third forming tool 30 and fourth forming tool 40, without substantial damage. Defining edges of any fluid exit ports provided may be radiused or rounded, which can help ease removal of second formation 102 from third forming tool 30 by mitigating any resistance to removal that may be caused by any stray fibers that may have extended into such from the main portion of first formation 100 during the compression step described above. As a result of its further compression and re-formation by third and fourth forming tools 30 and 40, upon its removal from third forming tool 30 and/or fourth forming tool 40, second formation 102 is imparted with a second formation angle α2′ that is similar or approximately equal to second forming angle α2. For the purpose of facilitating removal from the tools and any further formation steps that may be desired, it may be desired that carrier fluid removal is controlled so that first formation 102 retains a weight percentage of carrier fluid (e.g., water) of 15 percent to 25 percent.

It has been learned that the steps described above may be sufficient for imparting a second formation 102 that is to be a tampon applicator barrel blank, to be converted to a tampon applicator barrel, with a second formation angle α2′ that is not zero, but very nearly zero, such that the structure assumes a shape that is nearly cylindrical. This has been demonstrated with prototype tooling in first formation angle α1 imparted to the first forming surface 10a was 3 degrees, and the second formation angle imparted to the third forming surface 30a was 0.5 degrees.

It is contemplated, however, that in some examples a further step of compression and re-formation may be included in the process, depending upon the extent of and details included in the desired re-formation. In such examples, a pair of respective fifth and sixth forming tools (not shown) having added or changed features and/or changed forming angle may be used, to effect further compression and re-formation step in a manner similar to that described above.

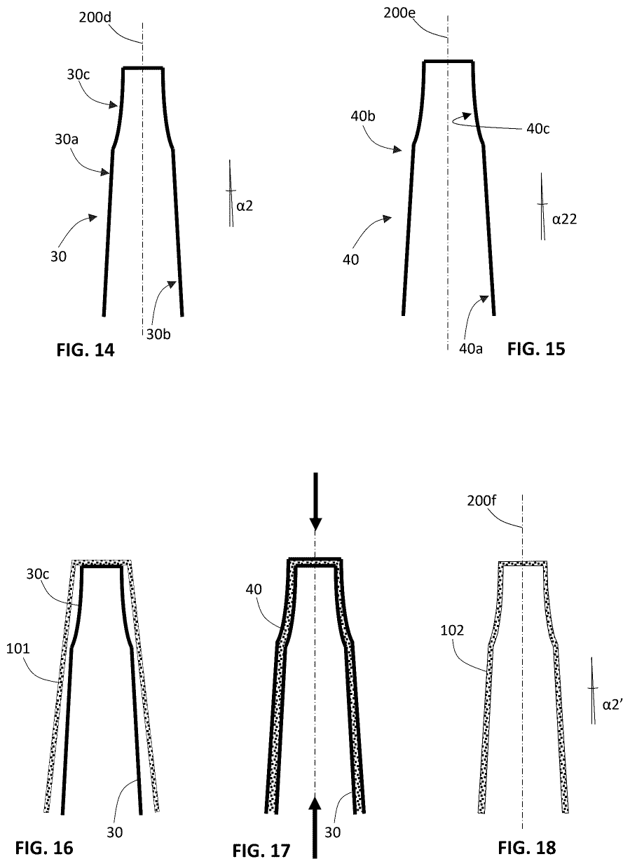

In another example, referring to FIGS. 14-18, a third forming tool 30 and fourth forming tool 40 may have features in addition to a changed (reduced) second forming angle α2. In the depicted example, third and fourth forming tools 30, 40 may include respective portions of changed shape 30c, 40c, to be imparted to the first formation 101, to yield second formation 102. Where the formed structure is to be converted to a tampon applicator barrel, for example, the changed shape may be, for example, a gripping portion of the barrel (portion at which a user of the tampon product will grip the applicator).

Preferably, the final formation following all forming/compressing/drying steps, will have a weight fraction of carrier fluid (e.g., water) of about 2 percent to about 8 percent, preferably to about 6 percent or even 5 percent, for purposes of stability of the final formation.

Preferably, one or more of first, second, third, fourth, etc., forming tools 10, 20, 30, 40, etc., respectively, are formed of metal or metal alloy such as stainless steel, such as will resist corrosion and withstand temperatures elevated above the boiling point of water at ordinary atmospheric pressure (e.g., temperatures up to 400 C), without becoming prone to softening and deforming in heating and compressing steps contemplated herein.

First, second, third, fourth, etc., forming tools 10, 20, 30, 40, etc., may be formed via any suitable machining process, or via additive manufacturing. For present purposes, and particularly with respect to first forming tool 10, it has been learned that additive manufacturing may be preferred, where, as contemplated herein, formed features of first forming tool 10 are geometrically complex, numerous and relatively small (e.g., fluid passage ports in a pattern), such that conventional (subtractive) machining processes may be difficult, slow and/or inefficient. Equipment and technologies are available to enable additive manufacturing of parts such as a first forming tool 10 of a variety of metals including stainless steel of various alloys, and other suitable metals and alloys. Additive manufacturing using metals in the present circumstances may be performed via laser beam powder bed fusion techniques, electron beam powder bed fusion, binder jetting and sintering, and/or any other suitable metal additive manufacturing techniques. A forming tool manufactured via additive manufacturing, particularly one manufactured of a metal or metal alloy, will have structural characteristics resulting from the this method of manufacturing. Prior to further surface finishing, an object manufactured via additive manufacturing will have somewhat rough or sandpaper-like surfaces. Additionally, the internal structure, viewed under magnification, may reflect a granular structure, reflected that particles of component material have been fused together. For this reason, it may be desired to provide machine surface finishing to at least the forming surface of a forming tool manufactured via additive manufacturing, to impart a smooth surface finish to the forming tool so as to reduce the chance for sticking between the tool and the structure being formed, and to impart a smooth surface finish to the formed structure. In some circumstances such a forming tool may have a smooth finish on the forming surface, while the internal surfaces of the fluid ports therein may retain their as-formed, rough surface finish.

In some examples, one or more of first forming tool 10, second forming tool 20, third forming tool 30, fourth forming tool 40, etc., may be provided with stick-resistant coating(s), such as, for example, coating(s) comprising polytetrafluoroethylene (a commonly known brand thereof is TEFLON) or boron nitride. Such coating(s) may serve to facilitate removal of the structure under formation, from the respective forming tools, in the forming steps described above, and help prevent damage to the structure during such removal.

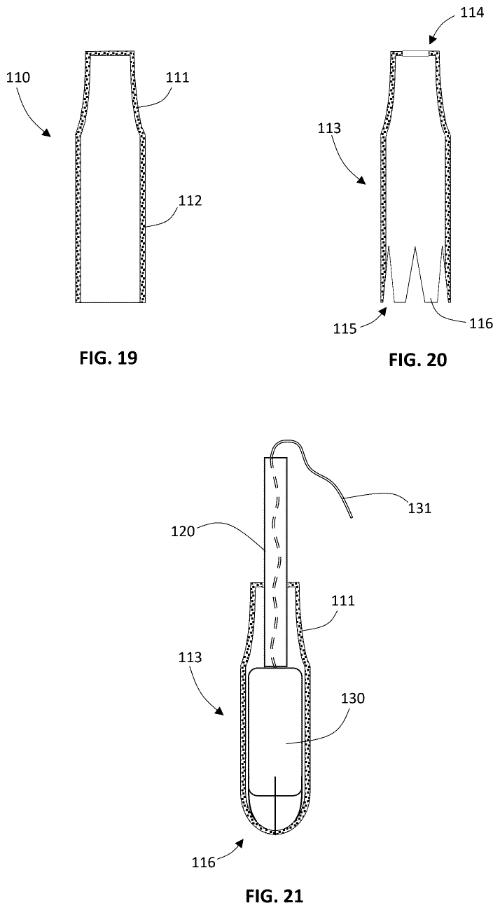

Referring now to FIGS. 19-21, the process described above may be used to manufacture a tampon applicator barrel blank 110, in some examples, having a grip portion 111 proximate a rearward end, and a main barrel portion 112 terminating at a forward end. Through downstream processes, in some examples blank 110 may be converted to an applicator barrel 113 by cutting out an ejection plunger passageway 114 and cutting out outlines 115 for petals 116 at the forward end. The barrel may be further formed by, e.g., application of heat and/or steam and compression on a forming mandril (not shown), to reshape the petals 116 into a curved, closed-end configuration as reflected in FIG. 21. FIG. 21 is a longitudinal cross section view of a completed tampon/applicator assembly, including applicator barrel 113, hollow ejection plunger 120, compressed tampon pledget 130, and withdrawal cord 131 attached thereto, respective configurations of which are known in various forms in the art (with the exception of the applicator barrel as described herein).

An applicator barrel manufactured as described herein has particular combination of characteristics. It is formed of a continuous agglomeration of fibers, unlike conventional paper or cardboard applicators that are formed of rolled and/or spiral-wound layers of paper. It will have no longitudinally-oriented seams of any substantial length, and preferably no seams, at which discrete previously-formed components have been joined and seamed together. It will have a substantially cylindrical appearance, but will not be perfectly cylindrical; it will have a formation angle (measured as described below) of greater than zero, but less than or equal to 3 degrees, more preferably less than or equal to 2 degrees, and even more preferably less than or equal to 1 degree. In some examples, it may be imparted with a wall caliper and/or wall basis weight that is greater at the rearward/gripping end, than at the forward/petal end. In some examples visible with aid of lighting and/or magnification, small, irregular and randomly located, but longitudinally-oriented, wrinkles 117 will be present and apparent, resulting from lateral/perimeter-wise compression as described above and reflected in FIGS. 25 and 26.

Additionally, as a characteristic result of such circumferential/hoop-wise compression and irregular formations of wrinkles or folds, the tampon applicator barrel particularly described herein, or other substantially conical or substantially cylindrical objects, will deviate from having perfect roundness about their circumferences. This deviation may generally increase, as the desired extent of progressive formation of the shape increases, from the initial compression between the first and second forming tools, to the final compression between the last forming tool used and the complementary next-to-last forming tool used. Extent of circularity/roundness (or lack thereof) in a substantially cylindrical or conical shape may be measured and expressed as a ratio of largest dimension to smallest dimension, divided by 2, where the largest and smallest dimensions are measured across a plane that is perpendicular to the longitudinal axis. A shape that is perfectly circular/round along this plane will have such a ratio of zero (0). Herein, a tampon applicator barrel or other substantially cylindrical or conical formed object may have a circularity/roundness of a value that is greater than zero, and less than or equal to 0.60, preferably less than or equal to 0.40, and more preferably less than or equal to 0.20. It may be appreciated that the circularity/roundness ratio for a substantially cylindrical or conical object formed as described herein may be reduced through attention to moisture content and extent of lateral/circumferential compression of the formation through the successive forming steps.

Formations of objects or features, other than near-cylindrically shaped objects such as tampon barrel blanks, formed as described herein, will similarly reflect formations of generally longitudinally-oriented wrinkles or folds, that will result from reduction in formation angle in successive steps, which forces the material to compress laterally as it is forced to conform with successive pairs of forming tools with successively decreasing forming angles.

### Formation Angle Measurement

Measuring a forming angle of a forming tool, or the formation angle of a formed object or formed feature as described herein, may be performed in any suitable manner. A longitudinal axis of a forming tool, formation, formed object or formed feature thereof is identified as described herein. The forming angle or formation angle is measured with respect to the identified longitudinal axis, and may be measured using any suitable method, via manual/visual or computer-assisted imaging and measurement techniques.

In view of the foregoing description, the following, non-limiting examples are contemplated herein:

### Method

1. A method for forming a structure comprising cellulosic fiber, comprising the steps of:
   providing a quantity of a slurry composition comprising cellulosic fibers suspended in a carrier fluid;
   providing a first forming tool having a first longitudinal axis and a wall having a first forming surface and a first non-forming surface, wherein the wall is perforated with a pattern of fluid passage ports extending from the first forming surface to the first non-forming surface; the pattern of fluid passage ports demarcates a surface area of the first forming surface; and at least a portion of the first forming surface forms a first forming angle α1 with the longitudinal axis of 1.5 to 5 degrees; wherein the ports are of a size effective to prevent passage of a weight majority of the cellulosic fibers therethrough;
   inundating the first forming surface with the slurry composition;
   either or both of:
applying vacuum to fluid in contact with the first non-forming surface; and/or applying positive pressure to the slurry composition in contact with the first forming surface,
   thereby drawing or urging the slurry composition to the first forming surface, and drawing or urging a portion of the carrier fluid in the quantity through the first pattern of ports, thereby forming a fiber agglomeration on the first forming surface;
   providing a second forming tool having a second longitudinal axis and a second forming surface, wherein the second forming surface is configured to fit into and/or about the first forming surface of the first forming tool in whole or in part, with a first clearance provided between the first forming surface and the second forming surface;
   placing the second forming surface into contact with the fiber agglomeration and into and/or about the first forming surface with the first and second longitudinal axes aligned and co-located, and applying opposing force along the longitudinal axes thereby applying pressure on the fiber agglomeration between the first forming surface and the second forming surface, whereby a first remaining portion of the carrier fluid is expressed from the fiber agglomeration, and whereby an impressed formed surface is formed on the agglomeration, whereby the agglomeration is transformed into a first formation having a first formation angle;
   providing a third forming tool having a third longitudinal axis and a third forming surface, the third forming surface forming a second forming angle with the third longitudinal axis that is less than one or both of the first forming angle and first formation angle;
   providing a fourth forming tool having a fourth longitudinal axis and a fourth forming surface, wherein the fourth forming surface is configured to fit into and/or about the third forming surface of the third forming tool in whole or in part, with a second clearance provided between the third forming surface and the fourth forming surface;
   removing the first formation from the first forming tool, and placing it into or onto the third forming tool;
   placing the fourth forming tool into contact with the first formation and into and/or about the third forming surface, with the third and fourth longitudinal axes aligned and co-located, and applying opposing force between the third forming tool and the fourth forming tool along the third and fourth longitudinal axes, thereby applying pressure on the first formation between the third forming surface and the fourth forming surface, whereby a second remaining portion of the carrier fluid is driven from the first formation, and whereby the first formation is transformed into a second formation having a second formation angle, and whereby lateral perimeter-wise compression is applied to the first formation and thereby imparts the second formation angle thereto, resulting in the second formation.
2. The method of example 1 wherein the second formation angle is less than the first formation angle.
3. The method of either of the preceding examples wherein the third forming surface and fourth forming surface have one or more shaping features that are in addition to any shaping features found on the first and second forming surfaces and in addition to a change in formation angle.
4 The method of any of the preceding examples wherein, following formation of the fiber agglomeration, heating energy is applied to one or more of the first forming tool, second forming tool, third forming tool and fourth forming tool, whereby carrier fluid is caused to evaporate from one or both of the fiber agglomeration and the first formation.
5. The method of any of the preceding examples wherein one or more of the second forming tool, third forming tool and fourth forming tool has/have one or more fluid exit ports from a forming surface thereof to a non-forming surface thereof, configured to allow carrier fluid and/or carrier fluid vapor to escape from the fiber agglomeration and/or the first formation.
6. The method of any of the preceding examples wherein a difference between the first forming angle and the second forming angle is 0.5 degrees to 4 degrees.
7. The method of any of the preceding examples wherein a difference between the first formation angle and the second formation angle is 0.5 degrees to 4 degrees.
8. The method of any of the preceding examples wherein the second formation angle is greater than zero degrees but equal to or less than 2 degrees, preferably equal or less than 1.5 degrees.
9. The method of any of the preceding examples wherein the first and third forming tools are male forming tools and the second and fourth forming tools are female forming tools.
10. The method of any of examples 1-8 wherein the first and third forming tools are female forming tools and the second and fourth forming tools are male forming tools.
11. The method of any of the preceding examples further comprising:
   providing a fifth forming tool having a fifth longitudinal axis and a fifth forming surface, the fifth forming surface forming a third forming angle with the fifth longitudinal axis that is less that the second forming angle α2;
   providing a sixth forming tool having a sixth longitudinal axis and a sixth forming surface, wherein the sixth forming surface is configured to fit into and/or about the fifth forming surface, with a third clearance provided between the fifth forming surface and the sixth forming surface;
   removing the second formation from the third forming tool, and placing it into or onto the fifth forming tool;
   placing the sixth forming surface into contact with the second formation and into and/or about the fifth forming surface, with the fifth and sixth longitudinal axes aligned and co-located, and applying opposing force along the fifth and sixth longitudinal axes between the fifth forming tool and the sixth forming tool, thereby applying pressure on the second formation between the fifth forming surface and the sixth forming surface, whereby a third remaining portion of the carrier fluid is driven from the second formation, and whereby the second formation is transformed into a third formation having a third formation angle, wherein the third formation angle is less than the second formation angle, and whereby lateral perimeter-wise compression is applied to the second formation and thereby imparts the third formation angle thereto, resulting in the third formation.
12. The method of example 11 wherein the fifth and sixth forming surfaces have one or more shaping features that are in addition to any shaping features found on the first, second, third and fourth forming surfaces and in addition to a change in formation angle.
13. The method of either of examples 11 or 12 wherein the third formation angle is greater than zero degrees but equal to or less than 2 degrees, preferably equal or less than 1.5 degrees.
14. The method of any of the preceding examples wherein the cellulose fibers have a weight composition predominantly of long fibers.
15. The method of example 14 wherein the long fibers comprise softwood fibers.
16. The method of any of the preceding examples wherein the softwood fibers comprise NSK fibers, SSK fibers, or a blend thereof.
17. The method of either of examples 15 or 16 wherein the softwood fibers comprise refined fibers.
18. The method of any of the preceding examples wherein the slurry comprises thermoplastic polymeric fibers or other synthetic fibers.
19. The method of example 18 wherein the synthetic fibers are hydrophilized prior to or during formation of the slurry composition.
20. The method of any of the preceding examples wherein the carrier fluid comprises predominantly water.
21. The method of any of the preceding examples wherein the first forming surface of the first forming tool has a dimension in the longitudinal direction equal to or greater than 5 mm.
22. The method of any of the preceding examples wherein the structure has an aspect ratio of dimension in a longitudinal direction, to greatest lateral dimension, of at least 1.5.
23. The method of any of the preceding examples wherein the first forming surface and the first formation have frusto-conical shapes.
24. The method of any of the preceding examples wherein the third forming surface and the second formation have frusto-conical shapes.
25. The method of any of the preceding examples wherein a last forming tool used has a last forming surface having a last forming surface area, wherein a majority, preferably the entirety of the last forming surface area, is without fluid exit ports.
26. The method of any of the preceding examples wherein a second-to-last forming tool used has a second-to-last forming surface having a second-to-last forming surface area, wherein a majority, preferably the entirety of the second-to-last forming surface area, is without fluid exit ports.
27. The method of any of the preceding examples wherein the second formation has a lower moisture content than the first formation.
28. The method of any of the preceding examples wherein a last formation has a lower moisture content than a preceding formation.
29. A structure manufactured using the method of any of the preceding examples.
30. The structure of example 29 wherein the structure is a tampon applicator barrel.

### Structure/Product

1. A structure comprising a formed continuous agglomeration of fibers including cellulose fibers, the structure having an outer surface having an outer shape with a longitudinal axis and a length measured along a direction parallel to the longitudinal axis, the outer surface having at least a portion lying at a formation angle relative the longitudinal axis that is greater than 0 degrees and equal to or less than less than 2 degrees, and preferably equal to or less than 1.5 degrees.
2 The structure of example 1 having an aspect ratio of length to greatest lateral dimension of 1.5 to 6.
3. The structure of either of the preceding examples wherein the portion of the outer surface lying at the formation angle has a longitudinal dimension greater than 10 mm.
4. The structure of any of the preceding examples wherein the structure is hollow, the outer surface is the outer surface of a wall, and the wall has a caliper and/or basis weight and/or density that varies along the length.
5. The structure of example 4 having a first end and a second end, wherein a caliper and/or basis weight and/or density proximate the first end is less than a caliper and/or basis weight and/or density proximate the second end.
6. The structure of any of the preceding examples, having no longitudinal seams present along a majority, and preferably the entirety, of the length.
7. The structure of any of the preceding examples having irregular and randomly-distributed, but generally longitudinally-oriented, wrinkles.
8. The structure of any of the preceding examples wherein at least a portion of the outer surface has a pyramidal profile.
9. The structure of any of the preceding examples wherein at least a portion of the outer surface has a conical profile.
10. The structure of any of the preceding examples wherein the structure is a tampon applicator barrel comprising the wall and having a forward end corresponding with the first end and an oppositely-disposed rearward end corresponding with the second end.
11. The structure of example 10 wherein the first end terminates in two or more petals.
12. The structure of any of examples 9-11 wherein the structure exhibits a roundness ratio of greater than zero, and equal to or less than 0.60, preferably equal to or less than 0.40, and more preferably equal to or less than 0.20.
13. The structure of any of the preceding examples wherein the fibers comprise thermoplastic polymeric fibers or other synthetic fibers.
14. The structure of example 13 wherein the synthetic fibers have been hydrophilized.

### Tooling

1. A forming tool for agglomerating thereon fibers from a slurry, the tool having a first end, a second end, a longitudinal axis, a forming surface and a non-forming surface, wherein the forming surface is perforated with a pattern of fluid passage ports extending from the forming surface to the non-forming surface; wherein the pattern of fluid passage ports demarks a surface area of the forming surface; and at least a portion of the forming surface forms a forming angle with the longitudinal axis of 1.5 to 5 degrees, preferably 1.5 to 4 degrees, and more preferably 1.5 to 3 degrees; wherein the ports are of a size effective to prevent passage of a weight majority of the fibers in the slurry therethrough.
2. The forming tool of example 1, wherein the pattern of fluid passage ports has a greater number of fluid passage ports per unit surface area at one or more first locations and a lesser number of fluid passage ports per unit surface area at one or more second locations.
3. The forming tool of example 2 wherein the pattern of fluid passage ports has a greater number of fluid passage ports per unit surface area at one or more first locations proximate the first end and a lesser number of fluid passage ports per unit surface area at one or more second locations proximate the second end.
4. The forming tool of any of the preceding examples, wherein fluid passage ports in the pattern have larger opening sizes at the forming surface at one or more first locations and smaller opening sizes at the forming surface at one or more second locations.
5. The forming tool of example 4 wherein the pattern of fluid passage ports have larger opening sizes at the forming surface at one or more first locations proximate the first end and smaller opening sizes at the forming surface at one or more second locations proximate the second end.
6. The forming tool of example 4, wherein fluid passage ports in the pattern have smaller opening sizes at the forming surface at one or more first locations proximate the first end and larger opening sizes at the forming surface at one or more second locations proximate the second end.
7 The forming tool of any of the preceding examples, wherein a plurality and preferably a majority of the fluid passage ports have relatively smaller opening sizes at the forming surface, and relatively larger opening sizes at the non-forming surface.
8. The forming tool of either of the preceding examples wherein a plurality and preferably a majority of the fluid passage ports have openings at the forming surface that lack sharp corners, and preferably, are elliptical, ovoid, oval, or circular in shape, and more preferably, are substantially circular in shape.
9. The forming tool of any of the preceding examples wherein openings of the fluid passage ports at the forming surface are defined by edges that are rounded or radiused.
10. The forming tool of any of the preceding examples, wherein a plurality and preferably a majority of the fluid passage ports on the at least a portion of the forming surface having forming angle, have center axes that form removal facilitating angles with respect to the longitudinal axis, wherein the removal facilitating angles are in a range of 15 degrees to 75 degrees, preferably 25 degrees to 65 degrees, and more preferably 35 degrees to 55 degrees.
11. The forming tool of any of the preceding examples, comprising stainless steel.
12. The forming tool of any of the preceding examples manufactured via additive manufacturing.
13. The forming tool of example 12, wherein the forming surface has been machined to impart a smooth surface finish thereon.
14. The forming tool of any of the preceding examples having a stick-resistant coating on the forming surface.
15. The forming tool of any of the preceding examples wherein the forming surface has a base shape that is circular or polygonal.
16. The forming tool of any of the preceding examples wherein the forming surface has a 3D shape that is at least in part conical or pyramidal.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A forming tool for agglomerating thereon fibers from a slurry, the tool having a first end, a second end, a longitudinal axis, a forming surface and a non-forming surface, wherein the forming surface is perforated with a pattern of fluid passage ports extending from the forming surface to the non-forming surface; wherein the pattern of fluid passage ports demarks a surface area of the forming surface; and at least a portion of the forming surface forms a forming angle (α1) with the longitudinal axis of about 1.5 to about 5 degrees; wherein the ports are of a size effective to prevent passage of a weight majority of the fibers in the slurry therethrough.

2. The forming tool of claim 1, wherein the pattern of fluid passage ports has a greater number of fluid passage ports per unit surface area at one or more first locations and a lesser number of fluid passage ports per unit surface area at one or more second locations.

3. The forming tool of claim 2 wherein the pattern of fluid passage ports has a greater number of fluid passage ports per unit surface area at one or more first locations proximate the first end and a lesser number of fluid passage ports per unit surface area at one or more second locations proximate the second end.

4. The forming tool of claim 1, wherein fluid passage ports in the pattern have larger opening sizes at the forming surface at one or more first locations and smaller opening sizes at the forming surface at one or more second locations.

5. The forming tool of claim 4, wherein the pattern of fluid passage ports have larger opening sizes at the forming surface at one or more first locations proximate the first end and smaller opening sizes at the forming surface at one or more second locations proximate the second end.

6. The forming tool of claim 4, wherein fluid passage ports in the pattern have smaller opening sizes at the forming surface at one or more first locations proximate the first end and larger opening sizes at the forming surface at one or more second locations proximate the second end.

7. The forming tool of claim 1, wherein a plurality of the fluid passage ports have relatively smaller opening sizes at the forming surface, and relatively larger opening sizes at the non-forming surface.

8. The forming tool of claim 7 wherein a plurality of the fluid passage ports have openings at the forming surface that are elliptical, ovoid, oval, or circular in shape.

9. The forming tool of claim 1 wherein openings of the fluid passage ports at the forming surface are defined by edges that are rounded or radiused.

10. The forming tool of claim 1, wherein a plurality of the fluid passage ports on the at least a portion of the forming surface having forming angle (al), have center axes that form removal facilitating angles with respect to the longitudinal axis, wherein the removal facilitating angles are in a range of about 15 degrees to about 75 degrees.

11. The forming tool of claim 1 comprising stainless steel.

12. The forming tool of claim 1 manufactured via additive manufacturing.

13. The forming tool of claim 12, wherein the forming surface has been machined to impart a smooth surface finish thereon.

14. The forming tool of claim 1 having a stick-resistant coating on the forming surface.

15. The forming tool of claim 1 wherein the forming surface has a base shape that is circular or polygonal.

16. The forming tool of claim 1 wherein the forming surface has a 3D shape that is at least in part conical or pyramidal.

17. A forming tool for agglomerating thereon fibers from a slurry, the tool having a first end, a second end, a longitudinal axis, a forming surface and a non-forming surface, wherein the forming surface is perforated with a pattern of fluid passage ports extending from the forming surface to the non-forming surface; wherein the pattern of fluid passage ports demarks a surface area of the forming surface; wherein a plurality of the fluid passage ports have relatively smaller opening sizes at the forming surface, and relatively larger opening sizes at the non-forming surface; and wherein the forming surface has a base shape that is circular or polygonal and a 3D shape that is at least in part conical or pyramidal.

18. The forming tool of claim 17, wherein at least a portion of the forming surface forms a forming angle (α1) with the longitudinal axis of about 1.5 to about 5 degrees.

19. The forming tool of claim 17, wherein a plurality of the fluid passage ports on the at least a portion of the forming surface having forming angle (α1), have center axes that form removal facilitating angles with respect to the longitudinal axis, wherein the removal facilitating angles are in a range of about 15 degrees to about 75 degrees.

20. The forming tool of claim 17 manufactured via additive manufacturing.
